(19) United States
(12) Patent Application Publication
(10) Pub. No.: US 2022/0308055 A1
(43) Pub. Date: Sep. 29, 2022

(54) METHOD AND DEVICE FOR PATHOGEN OR ANTIBODY DETECTION USING UNMODIFIED SIGNALING SUBSTANCES WITH OR WITHOUT UNLABELED DETECTION REAGENTS

(71) Applicants: UNIVERSITY OF CENTRAL FLORIDA RESEARCH FOUNDATION, INC., Orlando, FL (US); THE NEMOURS FOUNDATION, Jacksonville, FL (US); NANO DISCOVERY INC., Orlando, FL (US)

(72) Inventors: Qun HUO, Orlando, FL (US); Tianyu ZHENG, Orlando, FL (US); Kenneth Andrew ALEXANDER, Orlando, FL (US); Cedric PRITCHETT, Winter Park, FL (US); Peter E. PHELAN, Palm Bay, FL (US)

(21) Appl. No.: 17/639,023

(22) PCT Filed: Aug. 27, 2020
(86) PCT No.: PCT/US2020/048110

§ 371 Date: Feb. 28, 2022

Related U.S. Application Data
(60) Provisional application No. 62/892,258, filed on Aug. 27, 2019.

(51) Int. Cl. G01N 33/569

(52) U.S. Cl. CPC G01N 33/56983; G01N 2021/7786

(57) ABSTRACT

Disclosed herein are methods and devices for pathogen or antibody detection using unmodified signaling substances with or without unlabeled detection reagents. Unmodified signaling substance include, but are not limited to, a molecule, a material or a part of a material that is not modified with an antibody, an antigen, a pathogen, a DNA probe or an aptamer molecule. Unlabeled detection reagent refers to an antibody, an antigen, a pathogen, a DNA probe, or an aptamer that is not labeled with a signaling molecule such as enzyme, biotin, streptavidin, fluorescence or chemiluminescence probe, etc.

### BACKGROUND

The ability to accurately and efficiently detect, quantify, distinguish, and/or characterize analytes, including rare or small quantities of analytes, has notable benefits, including benefits in research and clinical settings. For example, analysis of biological molecules, such as cells, bacteria, viral particles, fungi, antibodies, complement, nucleotide and polypeptide molecules, or disease biomarkers in biological samples, has significant utility for diagnosis, prognosis, and/or treatment monitoring for a number of conditions. Indeed, the early diagnosis and treatment of many conditions can significantly increase survival rates for many life-threatening conditions.

The detection of analytes indicative of disease or infection in bodily fluids, such as blood, plasma, saliva, and urine, has several advantages when compared to other methods. The analysis of the presence of analytes in bodily fluids is a non-invasive method. Meanwhile, other convention methods of disease detection, such as tissue extraction (e.g. biopsy) is often a painful, expensive, and time-consuming procedure that can lead to complications. Accordingly, there remains a need for quick and inexpensive methods of detecting analytes in biological samples

### BRIEF DESCRIPTION OF THE DRAWINGS

The following drawing forms part of the present specification and is included to further demonstrate certain embodiments of the present invention. The invention may be better understood by reference to the drawing in combination with the detailed description of specific embodiments presented herein.

FIG. 1. provides a diagram illustrating an example of CMV and inclusion body detection from urine samples.

FIG. 2. provides a photograph relating to test results of 19 urine samples for CMV using the method illustrated in FIG. 1.

FIG. 3. provides a dark field optical microscope image of CMV inclusion bodies observed from a positive urine sample.

### DETAILED DESCRIPTION

Disclosed herein are methods and devices for pathogen or antibody detection using unmodified signaling substances with or without unlabeled detection reagents. Unmodified signaling substance include, but are not limited to, a molecule, a material or a part of a material that is not modified with an antibody, an antigen, a pathogen, a DNA probe or an aptamer molecule. Unlabeled detection reagent refers to an antibody, an antigen, a pathogen, a DNA probe, or an aptamer that is not labeled with a signaling molecule such as enzyme, biotin, streptavidin, fluorescence or chemiluminescence probe, etc. According to certain embodiments, the detection of a target pathogen or antibody is achieved by measuring or observing the property of the signaling substance upon non-specific interaction with at least one molecular component from the sample or from the mixture of a sample and detection reagent, if a detection reagent is used.

Most existing immunoassays require the labeling or conjugation of a detection reagent with a signaling molecule or substance to generate detectable signals. Such processes increase the time and cost required to develop and perform the assay. The disclosed methods and device described herein significantly reduce the cost of immunoassays, reduce the assay time, and enables the detection of a much broader range of pathogen and antibody targets.

According to one embodiment, provided is a method of detecting a pathogen in a sample that involves contacting the sample with an unlabeled detection reagent first, then contacting a signaling substance; or contacting the sample, the unlabeled detection reagent and the signaling substance concurrently all together. At least one molecular component derived from the sample or from the detection reagent, or a complex of both at least one molecular component from the sample and from the detection reagent, interacts with the signaling substance; and a property of the signaling substance is observed. A pathogen is detected based on the observed property.

It is noted that observing a property may include measuring the property. Further, detection of the pathogen may include comparing the observed property with a property of the signaling substance recorded from positive samples and negative samples. The method may include determining a presence, absence and/or quantity of the pathogen is determined.

In a specific embodiment, the method involves observing a property of the signaling substance before the signaling substance contacts the sample and/or unlabeled detection reagent and again after the signaling substance contacts the sample and/or unlabeled detection reagent.

In another related embodiment, the method involves observing a property of the signaling substance after contacting the sample, the unlabeled detection reagent and the signaling substance concurrently all together.

In specific examples, signaling substance implemented may pertain to nanoparticles, microparticles, magnetic particles, silica particles, polymer beads, quantum dots, electrodes, graphene, carbon nanotubes, or gold thin films, or a combination thereof.

According to another embodiment, provided is a method of detecting an immune response to a pathogen in a subject. The method may involve the steps of a) obtaining a sample from the subject; b) contacting the sample with an amount of the target pathogen under conditions to allow binding of at least one molecular component in the sample with the target pathogen; c) contacting the product of b) with a signaling substance; and d) observing a property of the signaling substance, wherein the pathogen is detected based on the observed property. In one embodiment, the sample is whole blood, blood plasma or blood serum. In a specific example, the at least one molecular component is an antibody.

In yet another embodiment, disclosed is a method of detecting a pathogen in a sample, wherein the pathogen produces inclusion bodies or protein aggregates in a subject. The method involves mixing a signaling substance with the sample under conditions to allow the signaling substance to interact with an inclusion body or protein aggregate in the sample; and observing a property of the signaling substance, wherein the pathogen is detected based on the observed property. In a specific embodiment, the pathogen is a virus. In another embodiment, the subject is a human or non-human mammal. In a further specific embodiment, the pathogen is cytomegalovirus (CMV). Inclusion boides produced pathogens include but are not limited to one or more owl's eye related to cytometalovirus, Cowdry type B related polio and adenovirus, Warthin-Finkeldey bodies related measles, Negri bodies related to Rabies, Tones bodies related to Yellow fever, Cowdry type A bodies related to Herpes simplex virus and Varicella zoster virus.

Another embodiment pertain to a kit for detecting a pathogen, or an antibody against the pathogen, in a sample. The kit comprises a container or a supporting material that holds or supports the sample; a detection reagent; and an unmodified signaling substance. The container may pertain to a tube, vial, syringe, dish, bowl, beaker or cylinder. The container or supporting material may be preloaded with the detection reagent and packaged or sealed prior to use.

### Definitions

The term “antibody” as used herein refers to any type of antibody or antibody fragments produced towards a pathogen or a component of a pathogen. The antibody can be presented in crude form or purified form. An example of crude form is antisera. An example of purified form is IgG or IgM fraction of the antibody purified by affinity chromatography. The antibody can be monoclonal or polyclonal. A single antibody or a combination of multiple antibodies targeting different epitopes or different components of the pathogen may be used in the assay.

The term “aptamer” as used herein refers to an oligonucleotide that is capable of forming a complex with an intended target substance. The complexation is target-specific in the sense that other materials which may accompany the target do not complex to the aptamer. It is recognized that complexation and affinity are a matter of degree; however, in this context, “target-specific” means that the aptamer binds to target with a much higher degree of affinity than it binds to contaminating materials.

The term “detection reagent” as used herein refers to a substance or a mixture of substances added to a sample to detect the target pathogen or target antibody. In all the methods disclosed here for pathogen detection, reagent means a substance or mixture of substances that contain at least one molecule of antibody and/or aptamer. For antibody detection, detection reagent means a pathogen or a component of a pathogen, or a combination of components of pathogen to which the antibody can specifically bind.

The term “inclusion bodies”, also called elementary bodies, as used herein involve their common scientific meaning of nuclear or cytoplasmic aggregates of substances, usually proteins. Examples of inclusion bodies include but not limited to: owl's eye in cytometalovirus, Cowdry type B in polio and adenovirus, Warthin-Finkeldey bodies in measles, Negri bodies in Rabies, Torres bodies in Yellow fever, Cowdry type A bodies in Herpes simplex virus and Varicella zoster virus.

The term “pathogen” as used herein refers to a virus, bacteria, parasites, fungi, or any other known pathogens to humans and animals. Examples of virus include but not limited to cytomegalovirus, herpes viruses, hepatitis A, hepatitis B, hepatitis C virus, Zika virus, West Niles virus, BK virus, HIV, norovirus, rotavirus. Pathogen here can refer to a whole pathogen, a part of the pathogen such as viral capsid, a component of a pathogen such as envelope proteins, structural proteins, non-structural proteins, or multiple components of a pathogen, or pathogen-derived products during host infection such as inclusion body, proteins, protein aggregates, and other molecules released from the host cells.

The term “property” as used herein refers to any physical, electrical, chemical, magnetic and optical properties. One specific example of optical properties is the color or light scattering property of the signaling substance. One specific example of physical property is the average size when the signaling substance is a nanoparticle such as a gold nanoparticle. Electrical property may include conductivity.

The term “sample” as used herein refers to a sample to be analyzed for the presence of a pathogen or a specific antibody towards a known pathogen. A sample can be obtained from a subject or other sources. Examples of a “sample” include but are not limited to blood (whole blood, blood plasma or blood serum), cerebrospinal fluid (CSF), tissue homogenates, cell suspension, urine, saliva, nasal swab, semen, sputum, vaginal fluid, or other fluids obtained from a subject. Samples may also pertain to extracts from plants or meat, or washing fluids that have been applied to edible objects including vegetables, fruits, or meat, or other collection from a surface of an object. Samples may also comprise a sample from soil or bodies of water, or wastewater.

The term “signaling substance” as used herein refers to a molecule, a material or a part of a material that is to be mixed with a sample and/or a detection reagent to generate a signal for detection. Examples of such substances include but not limited to nanoparticles, microparticles, magnetic particles, silica particles, polymer beads, quantum dots, electrodes, graphene, carbon nanotubes, gold thin films, surface of a device such as surface acoustic wave device, optical interferometer. A specific example of nanoparticle is gold nanoparticle.

The term “subject” as used herein refers to animal. Typical examples of an animal include but are not limited to mammals. In specific embodiments, the subject is a human, dog, cat, cow, horse, pig, goat, sheep, rat, mouse, guinea pig, or a nonhuman primate.

The term “unlabeled detection reagent” as used herein refers to a detection reagent that is not labeled with a signaling molecule such as enzyme, biotin, streptavidin, fluorescence or chemiluminescence probe, etc.

The term “unmodified signaling substance” as used herein refers to a signaling substance that is not modified with an antibody, an antigen, a pathogen, a DNA probe, or an aptamer molecule. The signaling substance will react with at least one molecular component from the sample or the detection reagent through non-specific interactions such as physical adsorption. For example, antibody or serum proteins can adsorb non-specifically to a citrate gold nanoparticle. A protein or protein aggregate released from a pathogen can cause gold nanoparticle aggregate formation upon non-specific adsorption to the gold nanoparticle. The identity of this molecular component may or may not be known. Such non-specifically targeted interaction will trigger a detectable property change such as color change, light scattering intensity change, average particle size change, electrical conductivity change, magnetic property change, surface optical property change, fluorescence property change of the signaling substance. This signal is used to assess the presence of pathogen or pathogen-specific antibody in an unknown sample using one of the two methods as described in Embodiment 1.

### DETAILED DESCRIPTION OF EXEMPLARY EMBODIMENTS

### Embodiment 1

In one embodiment, the method comprises a one or two-step test for suspected pathogen detection in an unknown sample. In the first step, an unlabeled antibody reagent that is specific to a target pathogen is mixed with a sample solution that contains the suspected target pathogen. The mixed solution is incubated for a certain time to allow interaction between the antibody and the pathogen. In a second step, this reaction product is mixed with a signaling substance. At least one molecular component from the reaction product will interact with the signaling substance. This molecular component can be from the sample or from the antibody reagent. After certain incubation time, the property of the signaling substance is observed or measured.

As an alternative to the above two-step test process, the reaction between the antibody and pathogen, and the signaling substance may be combined into a one-step process, which means, the sample, the detection reagent, and the signaling substance may be mixed altogether once to allow all reactions to occur.

Following the observation or measurement of the property from the signaling substance as a result of the above assay process, two methods are then used to determine the presence, absence and/or quantity of the suspected target pathogen: In method 1, the property of the signaling substance is recorded from known samples with (positive) and without (negative) the target pathogen. By comparing the property of the signaling substance obtained from an unknown sample to the property of known samples, the presence, absence and/or quantity of the suspected target pathogen is determined. In method 2, a second test in addition to the above test is performed. In this second assay, a sample solution is mixed with the signaling substance directly, without first reacting with the antibody reagent. The property difference of the signaling substance between the sample with and without reacting with the antibody reagent is compared and the difference is used to determine the presence, absence, and/or quantity of the suspected pathogen.

### Embodiment 2

In a second embodiment, the same tests as described in Embodiment 1 can be used to detect pathogen-specific antibody from blood, blood plasma, blood serum or other biological fluids using an unlabeled pathogen. While the assay process is the same as Embodiment 1, the antibody is now the target analyte to be detected from the sample, and the pathogen is now used as the detection reagent to detect the antibody from the sample. For example, to determine if a subject is infected with Zika virus and producing Zika-specific antibody in the blood, a blood serum or plasma taken from the subject is mixed with a solution containing a known identity and quantity of Zika virus. Following the same assay processes as described in Embodiment 1, a positive response will confirm that the subject is seropositive in Zika infection.

### Embodiment 3

In a third embodiment, a sample is mixed with an unmodified signaling substance to detect the presence of inclusion body, also called elementary body associated with a pathogen or a cell. In a specific example, inclusion body refers to inclusion body associated with cytomegalovirus (CMV). Inclusion body from CMV is a protein aggregate. Such protein aggregates can interact with an unmodified citrate-gold nanoparticle through non-specific interaction, causing gold nanoparticle to form aggregates. The property change of the gold nanoparticles, such as color change, light scattering change, or average particle size change, can be observed or measured to determine the presence, absence and/or quantity of CMV inclusion body in a sample. Other nanoparticles and microparticles such as polymer beads and magnetic beads may be used for the same purpose. This embodiment does not require the use of detection antibody reagent for the inclusion body detection. However, a second assay may be performed using a detection antibody and the process as described in Embodiment 1 to confirm the identity of the inclusion body.

Other examples of inclusion body include but not limited to: Negri bodies in Rabies, Guarnieri bodies in vaccinia and small pox, Cowdry type A in Herpes simplex virus and Varicella zoster virus, Cowdry type B in Polio and adenovirus, Torres bodies in yellow fever, etc.

For all of the above embodiments, the following variations may be applied. For pathogen detection, the antibody may be added to the sample in the form of a solution after the sample is added to an assay container or support, or the antibody can be pre-coated or placed in a container or support that will receive a sample solution later. Likewise, when pathogen is used for antibody detection, the pathogen may be added to the sample in the form of a solution after the sample is added to an assay container or support, or pre-coated or placed in a container or support that will receive a sample solution later. Similarly, the signaling substance may be added following the addition of detection reagent or sample or both into an assay container or support, or may be pre-coated or placed in a container or support that will receive a sample solution later. As an additional alternative, both signaling substance and the detection reagent, that is, either a pathogen reagent or antibody reagent, may be pre-coated or co-placed in a container or support that will receive a sample solution later.

### EXAMPLES

### Example 1: Detection of Cytomegalovirus and its Inclusion Bodies from Urine

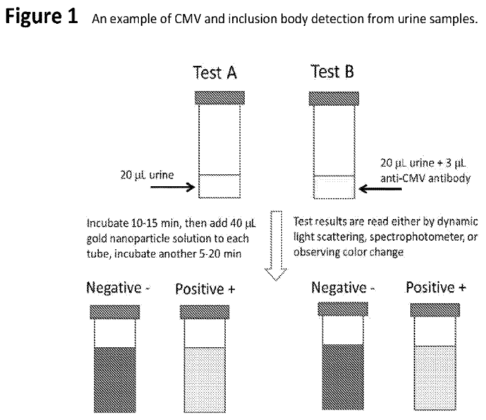

FIG. 1 is an illustration of a two-test assay (test A and test B) using the disclosed methods (Embodiment 1 and 3) for cytomegalovirus (CMV) detection from urine sample. In the first step, two portions of 20 μL urine sample from a subject is added to two containers, respectively. To one container, here is container B, 3 μL of anti-CMV antibody was added. Both containers are allowed to stand for 10-15 min. This time can vary from one antibody to another. Then to each container, 40 μL of a citrate-capped gold nanoparticle solution is added. After incubating for 5-20 min, the test result is read either by measuring gold nanoparticle size using dynamic light scattering, by a spectrophotometer, or by observing the color change or light scattering change of the assay solution through naked eyes.

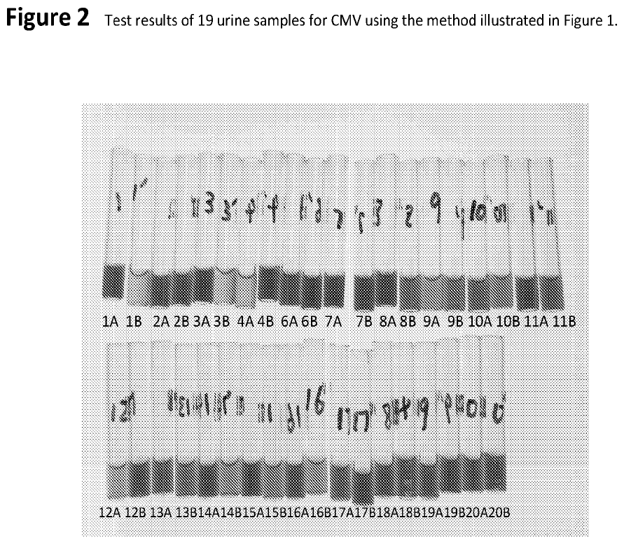

FIG. 2 is the color of the assay solution observed from 19 urine samples after performing the above test. Two tubes are used for each sample, with the first tube corresponding to test A and second tube corresponding to test B. If one of the two tests is positive, the sample is positive with CMV. Otherwise, sample is considered as negative. Sample #4 and #9 and #12 is tested positive in test A. Sample #1, #3, #8, #14, #15, and #16 is tested positive in test B. Therefore, sample #1, #3, #4, #8, #9, #12, #14, #15, and #16 are positive samples.

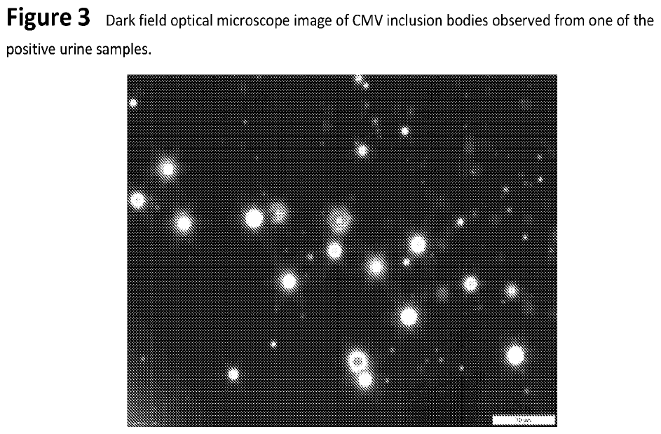

In this specific example, if test A is positive, it means the sample contains CMV-related inclusion body. CMV infection is known to lead to release of inclusion bodies to urine. Inclusion body is protein aggregate formed during CMV infection of host cells. The CMV inclusion body is also called “owl's eye”. Such inclusion bodies may be observed under an optical microscope, with the shape of an owl's eyes. FIG. 3 is a dark field optical microscope image of sample #9. Inclusion bodies were observed from sample #4, #9, and #12. No inclusion body structures were observed from other urine samples. Protein aggregates are known to interact with citrate gold nanoparticles to lead nanoparticle aggregate formation. Upon interaction with gold nanoparticles, the inclusion body will lead to gold nanoparticle aggregate formation, and a color change of the gold nanoparticles from dark purple to very tint color. By comparing the test results of urine samples with inclusion body and urine samples without inclusion body, the color difference or average nanoparticle size difference of the assay solution can be used to determine if an unknown sample contains inclusion body or not. Additionally, a test B can also be used to confirm the presence of CMV inclusion body. In test B, when anti-CMV antibody reagent is added to the urine sample, the antibody reagent will inhibit the interaction between inclusion body and the gold nanoparticle. As a result, the assay solution of test B maintains its original dark purple color, giving a negative signal. For all three samples tested positive in test A, #4, #9, and #12, the results of test B are negative.

If test B is positive, the sample contains CMV virus and/or virus antigens. The binding of the anti-CMV antibody will lead to the formation of antibody-virus complex or complex between antibody and virus components such antigen proteins. Antibodies will also interact with the citrate gold nanoparticles. The cross reaction between antibody reagent, the virus or virus components, and the gold nanoparticle will thus lead to nanoparticle aggregate formation, changing the color of the nanoparticle assay solution. The comparison of the color change of samples with CMV and without CMV present in the sample can be used to determine whether a sample is positive or negative. For all positive samples as tested in test B, the result of test A, same test as test B but without the use of anti-CMV antibody, remains negative. This confirms that without the antibody, the sample containing CMV virus will not interact with the gold nanoparticle and cause the color change of the assay solution.

Although two tests, test A and test B are performed to detect CMV infection from urine samples in this specific example, each test may be performed individually without the need of performing the other test. Specifically, test A may be performed as a standalone test to detect CMV inclusion bodies in urine or saliva samples. Because of the presence of inclusion bodies in urine and saliva is relatively unique to CMV infection, a second confirmation test such as test B is not needed for screening purpose. Similarly, test B may be performed alone to detect CMV virus from urine samples without test A. As demonstrated in this study, urine samples without CMV present does not cause significant color change after reacting with anti-CMV antibody and the gold nanoparticle, therefore, the color change of the assay solution from test B can be used directly to determine the presence and quantity of CMV virus and virus antigens in the sample.

The gold nanoparticle aggregate formation can also be detected by measuring the average particle size of the assay solution at the end of the assay using dynamic light scattering (DLS). Table 1 is the assay data. The test results using DLS matches the test results determined by observing the color change of the assay solution.

TABLE 1
Test results of 20 urine samples using dynamic light
scattering to measure the average particle size of
the assay product. Neg-negative; Pos-positive.

| | | | Interpretation | | |
|---|---|---|---|---|---|
| Sample | Test A | Test B | | | Sample |
| ID | (nm) | (nm) | Test A | Test B | diagnosis |
| 1 | 59.65 | 229.1 | Neg | Pos | Pos |
| 2 | 71.7 | 85.8 | Neg | Neg | Neg |
| 3 | 59.55 | 195.8 | Neg | Pos | Pos |
| 4 | 350.65 | 94.9 | Pos | Neg | Pos |
| 5 | 97.4 | 80.4 | Neg | Neg | Neg |
| 6 | 81.55 | 68.6 | Neg | Neg | Neg |
| 7 | 78.2 | 78.9 | Neg | Neg | Neg |
| 8 | 80.1 | 167.5 | Neg | Pos | Pos |
| 9 | 395.5 | 130.2 | Pos | Neg | Pos |
| 10 | 99.05 | 98.7 | Neg | Neg | Neg |
| 11 | 78.25 | 135.7 | Neg | Neg | Neg |
| 12 | 131.2 | 67.6 | Pos | Neg | Pos |
| 13 | 82.9 | 81.7 | Neg | Neg | Neg |
| 14 | 59.8 | 369.1 | Neg | Pos | Pos |
| 15 | 62.95 | 261.9 | Neg | Pos | Pos |
| 16 | 76.65 | 312.4 | Neg | Pos | Pos |
| 17 | 78 | 63.2 | Neg | Neg | Neg |
| 18 | 78.45 | 68.7 | Neg | Neg | Neg |
| 19 | 73 | 66.2 | Neg | Neg | Neg |
| 20 | 76.25 | 72.2 | Neg | Neg | Neg |

### Example 2. Use of any of the Above Methods and Devices for Diagnostic Applications

1. to screen newborn babies for congenital CMV infection;
2. to screen pregnant women for CMV infection;
3. to detect CMV infection from organ transplant patients;
4. to monitor the treatment effect of anti-CMV drugs on the infected patients
5. To screen or diagnosis early stage dementia, Alzheimer's disease, or other neurological disorders and diseases caused by infections with different herpes viruses

### Example 3. Devices and Instructions to Perform the Above Assays and Results Interpretation

A device or kit that may be used in conjunction with the methods described herein may include a container or a supporting material that will hold or support the sample, the detection reagent and the unmodified signaling substance to perform any of the above assays.

A device comprises a container or a supporting material that will hold the sample and an unmodified signaling substance to perform any of the above assays. Examples of container include, but are not limited to, a tube, vial, syringe, dish, bowl, beaker or cylinder.

An example of supporting material includes a porous material. Another example of supporting material is the surface of a material. In another example, the supporting material and the signaling substance is the same. Instructions on how to use the disclosed methods and devices for performing the assay.

Although the present invention has been described in considerable detail with reference to certain preferred versions thereof, other versions are possible. Therefore, the spirit and scope of the appended claims should not be limited to the description of the preferred versions contained herein. [0103] The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C § 112, sixth paragraph. In particular, the use of “step of” in the claims herein is not intended to invoke the provisions of 35 U.S.C § 112, sixth paragraph.

What is claimed is:

1. A method of detecting a pathogen in a sample, the method comprising
contacting (i) the sample with (ii) an unlabeled detection reagent first, then contacting (iii) a signaling substance, or contacting (i) the sample (ii) the unlabeled detection reagent and (iii) the signaling substance concurrently all together; wherein at least one molecular component derived from the sample or from the detection reagent, or a complex of both at least one molecular component from the sample and from the detection reagent interacts with the signaling substance; and
observing a property of the signaling substance, wherein the pathogen is detected based on the observed property.

2. The method of claim 1, wherein
observing a property of the signaling substance before the signaling substance contacts the sample and/or unlabeled detection reagent and again after the signaling substance contacts the sample and/or unlabeled detection reagent.

3. The method of claim 1, wherein observing a property of the signaling substance occurs after contacting (i) the sample (ii) the unlabeled detection reagent and (iii) the signaling substance concurrently all together.

4. The method of claim 2, wherein the signaling substance comprises nanoparticles, microparticles, magnetic particles, silica particles, polymer beads, quantum dots, electrodes, graphene, carbon nanotubes, or gold thin films, or a combination thereof.

5. The method of claim 1, wherein observing comprises measuring the property.

6. The method of claim 1, wherein detection of the pathogen comprises comparing the observed property with a property of the signaling substance recorded from positive samples and negative samples.

7. The method of claim 6, wherein a presence, absence and/or quantity of the pathogen is determined.

8. A method of detecting an immune response to a pathogen in a subject, the method comprising:
a) obtaining a sample from the subject;
b) contacting the sample with an amount of the target pathogen under conditions to allow binding of at least one molecular component in the sample with the target pathogen;
c) contacting the product of b) with a signaling substance; and
d) observing a property of the signaling substance, wherein the pathogen is detected based on the observed property.

9. The method of claim 8, wherein the sample is whole blood, blood plasma or blood serum.

10. The method of either claim 8 or 9, wherein the at least one molecular component is an antibody.

11. A method of detecting a pathogen in a sample, wherein the pathogen produces inclusion bodies or protein aggregates in a subject, the method comprising:
a) mixing a signaling substance with the sample under conditions to allow the signaling substance to interact with an inclusion body or protein aggregate in the sample; and
b) observing a property of the signaling substance, wherein the pathogen is detected based on the observed property.

12. The method of claim 11, wherein the pathogen is a virus.

13. The method of claim 11 or 12, wherein the subject is a human or non-human mammal.

14. The method of any of claims 11-13, wherein the pathogen is cytomegalovirus (CMV).

15. The method of any of claims 11-14, further comprising mixing with the sample, either before or after step (a), a labeled or unlabeled detection reagent.

16. The method of any of claim 11-13 or 15, wherein the inclusion body comprises one or more owl's eye related to cytomegalovirus, Cowdry type B related polio and adenovirus, Warthin-Finkeldey bodies related measles, Negri bodies related to Rabies, Torres bodies related to Yellow fever, Cowdry type A bodies related to Herpes simplex virus and Varicella zoster virus.

17. The method of claim 14, wherein the sample is urine.

18. A kit for detecting a pathogen, or an antibody against the pathogen, in a sample; the kit comprising a container or a supporting material that holds or supports the sample; a detection reagent; and an unmodified signaling substance.

19. The kit of claim 18, for use in performing a method according to any of claims 1-17.

20. The kit of claim 18, wherein the container comprises a tube, vial, syringe, dish, bowl, beaker or cylinder.

21. The kit of any of claims 18-20, wherein the container or supporting material is preloaded with the detection reagent and packaged or sealed prior to use.
